(19) United States
(12) Patent Application Publication
(10) Pub. No.: US 2004/0267235 A1
(43) Pub. Date: Dec. 30, 2004

(54) Pressure injection with minimum boundary deformation and/or stress change

(76) Inventor: William E. Hodge, Lumby (CA)

Correspondence Address: Fletcher Paine Associates Ltd., 2250 - 11th Avenue, Vernon, BC, V1T 7X8, CA

(21) Appl. No.: 10/836,216

(22) Filed: May 3, 2004

Related U.S. Application Data
(60) Provisional application No. 60/468,169, filed on May 6, 2003.

(51) Int. Cl. A61M 31/00

(52) U.S. Cl. 604/500

(57) ABSTRACT

A method and associated apparatus are given for permitting fluid of any viscosity to be Injeted into a delicate cavity or body space at virtually any rate of flow while minimizing the potentially deleterious deformations of the cavity walls which would otherwise accompany the rapid injection of a non-compressible fluid. The apparatus allows for the transmission of vibrations through the cavity fluid. It also offers the potential for dislodging and removing solids from the cavity by utilizing the hydraulic drag forces associated with relatively high fluid velocities safely attainable using this method and apparatus.

### CROSS-REFERENCE TO RELATED APPLICATIONS

This application claims priorty under 35 U.S.C 119(e) to U.S. Provisional Patent application No. 60/468,169 filed May 6, 2003, the disclosure of which is incorporated herein by reference. Formerly the Invention was referred to as a “CONSTANT SPACE VOLUME INJECTOR”. Subsequently the title was changed to better reflect the function of the method.

### BACKGROUND OF THE INVENTION

1. Field of Invention

The method and apparatus described herein is intended to generally facilitate and improve the ability to replace a fluid within a confined space by another fluid while causing minimal change to the volume, shape, and ambient pressure of that space. It is intended for use in medical procedures, but may also have application in industry and engineering where it is necessary to maintain the boundary integrity of a space while injecting a fluid into that containment. For convenience of presentation, the elucidation of the novelty will be in terms of medical intervention.

2. Description of Related Art

Current medical practice involves injecting fluids into body spaces using a hypodermic needle, and at best, on some occasions, allowing the excess body fluid to vent to the atmosphere by bleeding from a second tube installed into the same space. Using that method and procedure, in order to make an external fluid flow into a confined space, such as a body vessel or cavity, it is necessary to exert a pressure on the fluid sufficient to overcome hydraulic resistance within the total system and allow venting to atmospheric pressure. This results in an increase in pressure within the confined space which is accompanied by volumetric dilation and the concomitant tensioning of the confinement boundary. This distortion can lead to rupture at the site, or at any weak point within the body which is affected by the change.

### SUMMARY OF THE INVENTION

### Disclosure of the Invention

The method advocated here is simply to take and conduct all the fluid displaced by bleeding from the confined space and direct it into the cylinder behind the advancing plunger. The system is thereby constrained to becoming one of constant volume. Consequently, by providing an apparatus which is relatively rigid, movements of the plunger can not result in volume change within the confined space. Without volume or shape change of the confined space there can be no additional stressing of the containment boundary and therefore no structural distress.

### OBJECTS AND ADVANTAGES

Because the containment vessel will suffer no additional stressing, irrespective of the rate of plunger movement, the injection process can be carried out safely at any speed. The only consequence of quantity rate transfer is increase in net pressure application at the plunger. Velocities within the space may be increased to enable non-fluid masses within the contained space to be eroded and transported out of the system.

### BRIEF DESCRIPTION OF THE DRAWINGS

Aspects of the invention are illustrated, merely by way of example, in the accompanying drawing in which:

List of Reference Numerals

10 confined space such as body cavity

20 cylindrical injection device

21 fluid on input side of cylinder

22 fluid on drainage side of cylinder

23 tap to saturate input side of cylinder

24 conduit conveying fluid to cavity

25 conduit conveying fluid from cavity

26 tap to flush drainage side of cylinder

27 seal between conduit and cavity

30 injector mechanism

31 plunger with iron core

32 mobile magnet

As will be obvious from this drawing, the procedure and apparatus required for engineering or non-medical purposes could readily be adapted to particular physical environments without departing from the fundamental novelty of this invention.

### DESCRIPTION OF A PREFERRED EMBODIMENTS

### Description of the Device

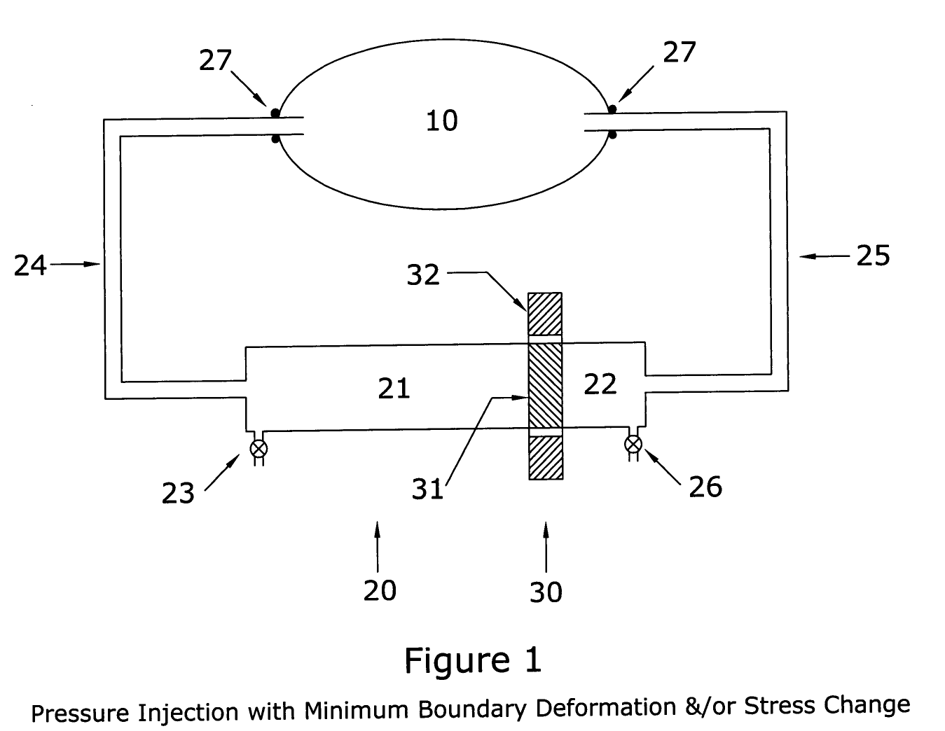

One feasible configuration of the apparatus is illustrated in FIG. 1, and is as follows:

A fluid filled cavity 10 is connected to a cylinder 20 by means of conduits 24 and 25 attached to the confined space 10 at positions selected to facilitate fluid exchange. Although the flow direction is reversible, for convenience of description, conduit 24 will here be considered to conduct flow into the cavity, and conduit 25 will be considered to convey discharge from the cavity back to the cylinder. As will be obvious the mechanism is entirely reversible.

The interconnection between the cavity and the conduits are made leakproof by seals 27. The system consisting of cavity, conduits, and cylinder is thereby made into a composite volume of fixed size. By constructing the cylinder and conduits of relatively rigid materials, with respect to the cavity ductility, provided leakage from the system is prevented, fluid exchange between the cylinder and cavity will take place without causing deformation of the cavity wall.

The cylinder 20 is separated into two compartments by a plunger 30 which is designed to prevent flow between the compartments. Fluid is caused to flow by movement of the piston 31. The piston in this configuration contains an imbedded iron core. Moving magnet 32, which is adjacent to the piston, but on the outside of the cylinder, will result in the plunger being made to translate in unison. The rate of piston translation, and consequently, the rate of fluid flow through the cavity, is dictated by the movements of the magnet 32. Rapid fluid flow within the cavity has the potential to cause solids within the cavity, or non-fluid adhesions to the cavity wall, to be dislodged by hydraulic drag forces. At sufficiently high fluid velocities such objects may be carried in suspension out of the cavity and into the cylinder compartment 22.

The magnet can be made to move at a range of speeds in either direction. Thus the plunger can be made to transmit vibrations to the fluid continuum by causing the magnet movement to be cyclic, with the characteristics of the vibrations being controllable by the frequency of the magnet oscillations. In this manner an organ can be massaged internally.

### Operation of Invention

The forward side of the cylinder 21 is filled with the fluid to be injected into the confined space. The forward conduit 24 is filled with a neutral fluid, or confined space fluid, and the open end is inserted into the confined space and sealed thereto. Tap 23 allows this saturation of the input side to be accomplished.

The venting conduit 25 from the confined space is connected to the trailing side of the cylinder 22 and that side of the system allowed to saturate by bleeding from the tap 26.

Once the cylinder and tubing on both sides of the system are fluid filled the piston is advanced at a rate either predetermined, or adjusted on the basis of monitoring of the confined space response. The fluid 21 on the piston side in front of the magnet advance is pushed into the confined space, while at the same time fluid 22 of an equal volume is drawn into the space trailing the advancing piston. In this manner the volume of the confined space remains unaltered during the procedure.

Pulsating the piston to agitate the fluids and vibrate the confined space, or surging the piston in reverse directions to “scrub” the cavity walls, are available options.

I claim:

1. A method of injecting a fluid into a fluid filled confined space so as to minimize deformation and additional stressing of the boundary of that space irrespective of the rate at which the fluid is introduced.

2. A method of injecting a fluid into a fluid filled confined space so as to flush non-fluid debris and adhesions from the space while minimize stressing and deformation of that space.

3. An apparatus for injecting a fluid under pressure into a body cavity or space so as to facilitate the rapid introduction of the fluid while minimizing shock to the patient and risk of damaging the cavity walls or adjacent organs.
